SEQUENCE LISTING (15 sequences)

SEQ ID NO: 1 - DNA, 3132 bp, Sorghum bicolor
cgcacacccaaaccccatcccgaacgcagaagatcccgtgcccgtgccagcggcggcgacggtgtgtgtggtgtggactggtagccatcatcaatccaggcatccccgtccacccgtaaccccgccgcccggcccccgcggacgccggcaatggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactggtacacctccacgattcccctgccgaactgtcctgttaagctttcaactcaggtctttgacttctgcttcatctctgctgtttgcagtggacacgggagcttctcaaacaaccccaagctcaacagcgtggtcggccacatactccattcctccattcttgtcccataccacggatggtaggattgcccatgtttcttacgcttgttactttgttagttagtttctgtcgaggattatcgttgatgttaaggaaggacctgtctttgaatggaattcaggaggattagccacaggacgcaccatcaaaaccacggccacgttgagaaggacgagtcctggcacccggtaggtttcctgtcgccctctcttcagttggagcttgagaagttatgctgatggtacatgtctatgaatggtgcttgatggttcctcggattgtgattacttgcagctaccagagaggctgtacaaaagcctggactttatgactaagaaattgcggttcaccatgccattccccctgctcgcgttcccgttatacttggtgaggctaagagcatctctattgtatgatacatgaattggacgaataaatgttttttgttttatggcgtggtagtggtgctaaattcctgccctgtgggttcatgattatgtagttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtaagagcgacatgtcctcctcttcagacccaacttctgcaaaaggataatttgaatgggtgatgtctgtgtgctcgaaatttatgtggctgctgctggtgtgtgcaggtatttgttgcttggctggatatggtcacatacttgcaccatcatggccacgaagacaagcttccctggtaccgtggaaaggtgattattagcaaagcaattttccaatttgttgatgtttcaaggtcccagtaatcttaaaaaactacagttacttccgtttttggttggttagaagcttttaaatgtttgcctcagctaagtcaggacgttattaattgcagcgaattgttgaatcataatacatcctatcctagaaatggaagttgtgtaaggactaaggacacaccatgccagaaatgttagatgcgcaatgacttcctctccttttgatttaccagtattttggataaaactggctgctttccattgcgccacttgacttcagcatctgtaggctatctgttcttgctggtagatacatgtttggtagactgatagacttcaacatctggactcactttgtttgcaaatggctagcttgaccttgaaacctttaaatcatatataaaccctagtggtggatattacaaaccaaagttctatccttggtaattttctggagtaagatctaattggaggatgctattgaactattggacaaaatgtctatttctcttttgtctttatgaggggaaatgatgtaactttgtatttcgtcgcttaccgagtgtaacgctaggtcagcctattatatagttttcagttttcactttacttttcagattatcgattgctgttaatttataagtattctatcctgatttctccttttgaagatagattggttattgataccacattgtccttttctatattaggaatggagttatctgcgtggaggactgacgacactcgaccgggactatggattgatcaacaatatccatcatgacattggaacccatgtcatccatcaccttttccctcaaatcccacattaccatctcattgaggcggtaagagtttcccctattcttgcctttttttcatttttattataagtttctttgcctagatcataagacctgttcttggttgacctgcagactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattgacacggactatagaacgtagcgtattcttcatttttctcaggggggctgtgcctgaagacctgcccagcctcccgggacctctgattggtgtgacttggtacacgtcaattcaatcaagaatgtaaggtggcataagaattatcatgtatgggagggtaaatgtagttctttccaatgatggaaaacggatgctgcttccgatgtaacaacatgtgattctttggactagaggtatactatttacagtaaagagtgaaaaaaaaggacttagctacatg

SEQ ID NO: 2 - DNA, 1356 bp, Sorghum bicolor
atggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactgtggacacgggagcttctcaaacaaccccaagctcaacagcgtggtcggccacatactccattcctccattcttgtcccataccacggatggaggattagccacaggacgcaccatcaaaaccacggccacgttgagaaggacgagtcctggcacccgctaccagagaggctgtacaaaagcctggactttatgactaagaaattgcggttcaccatgccattccccctgctcgcgttcccgttatacttgttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtatttgttgcttggctggatatggtcacatacttgcaccatcatggccacgaagacaagcttccctggtaccgtggaaaggaatggagttatctgcgtggaggactgacgacactcgaccgggactatggattgatcaacaatatccatcatgacattggaacccatgtcatccatcaccttttccctcaaatcccacattaccatctcattgaggcgactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattga

SEQ ID NO: 3 - protein, 451 aa, Sorghum bicolor
MARLVLSECCGLAPLRLRAGRRGAAIAAPSPAALSVAAAAVVPGRPASAAIHRDWALRVSAPTRLTSAVEEDKRSSPLGEEGVADSGAAGGDGFDPGAPPPFGLAEIRAAIPKHCWVKDPWRSMSYVLRDVVVVLGLAAAAARLDSWLVWPLYWAAQGTMFWALFVLGHDCGHGSFSNNPKLNSVVGHILHSSILVPYHGWRISHRTHHQNHGHVEKDESWHPLPERLYKSLDFMTKKLRFTMPFPLLAFPLYLFARSPGKTGSHFNPSSDLFQPNEKKDIITSTASWLAMVGVLAGLTFMMGPIPMLKLYGVPYLVFVAWLDMVTYLHHHGHEDKLPWYRGKEWSYLRGGLTTLDRDYGLINNIHHDIGTHVIHHLFPQIPHYHLIEATEAAKPVLGKYYKEPKKSGALPWHLFGVLAQSLKQDHYVSDTGDVVYYQTDSKTNTSAQKSD

SEQ ID NO: 4 - DNA, 3132 bp, ARTIFICIAL SEQUENCE
  CHEMICALLY SYNTHESIZED
cgcacacccaaaccccatcccgaacgcagaagatcccgtgcccgtgccagcggcggcgacggtgtgtgtggtgtggactggtagccatcatcaatccaggcatccccgtccacccgtaaccccgccgcccggcccccgcggacgccggcaatggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactggtacacctccacgattcccctgccgaactgtcctgttaagctttcaactcaggtctttgacttctgcttcatctctgctgtttgcagtggacacgggagcttctcaaacaaccccaagctcaacagcgtggtcggccacatactccattcctccattcttgtcccataccacggatggtaggattgcccatgtttcttacgcttgttactttgttagttagtttctgtcgaggattatcgttgatgttaaggaaggacctgtctttgaatggaattcaggaggattagccacaggacgcaccatcaaaaccacggccacgttgagaaggacgagtcctggcacccgataggtttcctgtcgccctctcttcagttggagcttgagaagttatgctgatggtacatgtctatgaatggtgcttgatggttcctcggattgtgattacttgcagctaccagagaggctgtacaaaagcctggactttatgactaagaaattgcggttcaccatgccattccccctgctcgcgttcccgttatacttggtgaggctaagagcatctctattgtatgatacatgaattggacgaataaatgttttttgttttatggcgtggtagtggtgctaaattcctgccctgtgggttcatgattatgtagttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtaagagcgacatgtcctcctcttcagacccaacttctgcaaaaggataatttgaatgggtgatgtctgtgtgctcgaaatttatgtggctgctgctggtgtgtgcaggtatttgttgcttggctggatatggtcacatacttgcaccatcatggccacgaagacaagcttccctggtaccgtggaaaggtgattattagcaaagcaattttccaatttgttgatgtttcaaggtcccagtaatcttaaaaaactacagttacttccgtttttggttggttagaagcttttaaatgtttgcctcagctaagtcaggacgttattaattgcagcgaattgttgaatcataatacatcctatcctagaaatggaagttgtgtaaggactaaggacacaccatgccagaaatgttagatgcgcaatgacttcctctccttttgatttaccagtattttggataaaactggctgctttccattgcgccacttgacttcagcatctgtaggctatctgttcttgctggtagatacatgtttggtagactgatagacttcaacatctggactcactttgtttgcaaatggctagcttgaccttgaaacctttaaatcatatataaaccctagtggtggatattacaaaccaaagttctatccttggtaattttctggagtaagatctaattggaggatgctattgaactattggacaaaatgtctatttctcttttgtctttatgaggggaaatgatgtaactttgtatttcgtcgcttaccgagtgtaacgctaggtcagcctattatatagttttcagttttcactttacttttcagattatcgattgctgttaatttataagtattctatcctgatttctccttttgaagatagattggttattgataccacattgtccttttctatattaggaatggagttatctgcgtggaggactgacgacactcgaccgggactatggattgatcaacaatatccatcatgacattggaacccatgtcatccatcaccttttccctcaaatcccacattaccatctcattgaggcggtaagagtttcccctattcttgcctttttttcatttttattataagtttctttgcctagatcataagacctgttcttggttgacctgcagactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattgacacggactatagaacgtagcgtattcttcatttttctcaggggggctgtgcctgaagacctgcccagcctcccgggacctctgattggtgtgacttggtacacgtcaattcaatcaagaatgtaaggtggcataagaattatcatgtatgggagggtaaatgtagttctttccaatgatggaaaacggatgctgcttccgatgtaacaacatgtgattctttggactagaggtatactatttacagtaaagagtgaaaaaaaaggacttagctacatg

SEQ ID NO: 5 - protein, 223 aa, ARTIFICIAL SEQUENCE
  CHEMICALLY SYNTHESIZED
MARLVLSECCGLAPLRLRAGRRGAAIAAPSPAALSVAAAAVVPGRPASAAIHRDWALRVSAPTRLTSAVEEDKRSSPLGEEGVADSGAAGGDGFDPGAPPPFGLAEIRAAIPKHCWVKDPWRSMSYVLRDVVVVLGLAAAAARLDSWLVWPLYWAAQGTMFWALFVLGHDCGHGSFSNNPKLNSVVGHILHSSILVPYHGWRISHRTHHQNHGHVEKDESWHP

SEQ ID NO: 6 - DNA, 3132 bp, ARTIFICIAL SEQUENCE
  CHEMICALLY SYNTHESIZED
cgcacacccaaaccccatcccgaacgcagaagatcccgtgcccgtgccagcggcggcgacggtgtgtgtggtgtggactggtagccatcatcaatccaggcatccccgtccacccgtaaccccgccgcccggcccccgcggacgccggcaatggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactggtacacctccacgattcccctgccgaactgtcctgttaagctttcaactcaggtctttgacttctgcttcatctctgctgtttgcagtggacacgggagcttctcaaacaaccccaagctcaacagcgtggtcggccacatactccattcctccattcttgtcccataccacggatggtaggattgcccatgtttcttacgcttgttactttgttagttagtttctgtcgaggattatcgttgatgttaaggaaggacctgtctttgaatggaattcaggaggattagccacaggacgcaccatcaaaaccacggccacgttgagaaggacgagtcctggcacccggtaggtttcctgtcgccctctcttcagttggagcttgagaagttatgctgatggtacatgtctatgaatggtgcttgatggttcctcggattgtgattacttgcagctaccagagaggctgtacaaaagcctggactttatgactaagaaattgtggttcaccatgccattccccctgctcgcgttcccgttatacttggtgaggctaagagcatctctattgtatgatacatgaattggacgaataaatgttttttgttttatggcgtggtagtggtgctaaattcctgccctgtgggttcatgattatgtagttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtaagagcgacatgtcctcctcttcagacccaacttctgcaaaaggataatttgaatgggtgatgtctgtgtgctcgaaatttatgtggctgctgctggtgtgtgcaggtatttgttgcttggctggatatggtcacatacttgcaccatcatggccacgaagacaagcttccctggtaccgtggaaaggtgattattagcaaagcaattttccaatttgttgatgtttcaaggtcccagtaatcttaaaaaactacagttacttccgtttttggttggttagaagcttttaaatgtttgcctcagctaagtcaggacgttattaattgcagcgaattgttgaatcataatacatcctatcctagaaatggaagttgtgtaaggactaaggacacaccatgccagaaatgttagatgcgcaatgacttcctctccttttgatttaccagtattttggataaaactggctgctttccattgcgccacttgacttcagcatctgtaggctatctgttcttgctggtagatacatgtttggtagactgatagacttcaacatctggactcactttgtttgcaaatggctagcttgaccttgaaacctttaaatcatatataaaccctagtggtggatattacaaaccaaagttctatccttggtaattttctggagtaagatctaattggaggatgctattgaactattggacaaaatgtctatttctcttttgtctttatgaggggaaatgatgtaactttgtatttcgtcgcttaccgagtgtaacgctaggtcagcctattatatagttttcagttttcactttacttttcagattatcgattgctgttaatttataagtattctatcctgatttctccttttgaagatagattggttattgataccacattgtccttttctatattaggaatggagttatctgcgtggaggactgacgacactcgaccgggactatggattgatcaacaatatccatcatgacattggaacccatgtcatccatcaccttttccctcaaatcccacattaccatctcattgaggcggtaagagtttcccctattcttgcctttttttcatttttattataagtttctttgcctagatcataagacctgttcttggttgacctgcagactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattgacacggactatagaacgtagcgtattcttcatttttctcaggggggctgtgcctgaagacctgcccagcctcccgggacctctgattggtgtgacttggtacacgtcaattcaatcaagaatgtaaggtggcataagaattatcatgtatgggagggtaaatgtagttctttccaatgatggaaaacggatgctgcttccgatgtaacaacatgtgattctttggactagaggtatactatttacagtaaagagtgaaaaaaaaggacttagctacatg

SEQ ID NO: 7 - protein, 451 aa, ARTIFICIAL SEQUENCE
  CHEMICALLY SYNTHESIZED
MARLVLSECCGLAPLRLRAGRRGAAIAAPSPAALSVAAAAVVPGRPASAAIHRDWALRVSAPTRLTSAVEEDKRSSPLGEEGVADSGAAGGDGFDPGAPPPFGLAEIRAAIPKHCWVKDPWRSMSYVLRDVVVVLGLAAAAARLDSWLVWPLYWAAQGTMFWALFVLGHDCGHGSFSNNPKLNSVVGHILHSSILVPYHGWRISHRTHHQNHGHVEKDESWHPLPERLYKSLDFMTKKLWFTMPFPLLAFPLYLFARSPGKTGSHFNPSSDLFQPNEKKDIITSTASWLAMVGVLAGLTFMMGPIPMLKLYGVPYLVFVAWLDMVTYLHHHGHEDKLPWYRGKEWSYLRGGLTTLDRDYGLINNIHHDIGTHVIHHLFPQIPHYHLIEATEAAKPVLGKYYKEPKKSGALPWHLFGVLAQSLKQDHYVSDTGDVVYYQTDSKTNTSAQKSD

SEQ ID NO: 8 - DNA, 3132 bp, ARTIFICIAL SEQUENCE
  CHEMICALLY SYNTHESIZED
cgcacacccaaaccccatcccgaacgcagaagatcccgtgcccgtgccagcggcggcgacggtgtgtgtggtgtggactggtagccatcatcaatccaggcatccccgtccacccgtaaccccgccgcccggcccccgcggacgccggcaatggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactggtacacctccacgattcccctgccgaactgtcctgttaagctttcaactcaggtctttgacttctgcttcatctctgctgtttgcagtggacacgggagcttctcaaacaaccccaagctcaacagcgtggtcggccacatactccattcctccattcttgtcccataccacggatggtaggattgcccatgtttcttacgcttgttactttgttagttagtttctgtcgaggattatcgttgatgttaaggaaggacctgtctttgaatggaattcaggaggattagccacaggacgcaccatcaaaaccacggccacgttgagaaggacgagtcctggcacccggtaggtttcctgtcgccctctcttcagttggagcttgagaagttatgctgatggtacatgtctatgaatggtgcttgatggttcctcggattgtgattacttgcagctaccagagaggctgtacaaaagcctggactttatgactaagaaattgcggttcaccatgccattccccctgctcgcgttcccgttatacttggtgaggctaagagcatctctattgtatgatacatgaattggacgaataaatgttttttgttttatggcgtggtagtggtgctaaattcctgccctgtgggttcatgattatgtagttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtaagagcgacatgtcctcctcttcagacccaacttctgcaaaaggataatttgaatgggtgatgtctgtgtgctcgaaatttatgtggctgctgctggtgtgtgcaggtatttgttgcttagctggatatggtcacatacttgcaccatcatggccacgaagacaagcttccctggtaccgtggaaaggtgattattagcaaagcaattttccaatttgttgatgtttcaaggtcccagtaatcttaaaaaactacagttacttccgtttttggttggttagaagcttttaaatgtttgcctcagctaagtcaggacgttattaattgcagcgaattgttgaatcataatacatcctatcctagaaatggaagttgtgtaaggactaaggacacaccatgccagaaatgttagatgcgcaatgacttcctctccttttgatttaccagtattttggataaaactggctgctttccattgcgccacttgacttcagcatctgtaggctatctgttcttgctggtagatacatgtttggtagactgatagacttcaacatctggactcactttgtttgcaaatggctagcttgaccttgaaacctttaaatcatatataaaccctagtggtggatattacaaaccaaagttctatccttggtaattttctggagtaagatctaattggaggatgctattgaactattggacaaaatgtctatttctcttttgtctttatgaggggaaatgatgtaactttgtatttcgtcgcttaccgagtgtaacgctaggtcagcctattatatagttttcagttttcactttacttttcagattatcgattgctgttaatttataagtattctatcctgatttctccttttgaagatagattggttattgataccacattgtccttttctatattaggaatggagttatctgcgtggaggactgacgacactcgaccgggactatggattgatcaacaatatccatcatgacattggaacccatgtcatccatcaccttttccctcaaatcccacattaccatctcattgaggcggtaagagtttcccctattcttgcctttttttcatttttattataagtttctttgcctagatcataagacctgttcttggttgacctgcagactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattgacacggactatagaacgtagcgtattcttcatttttctcaggggggctgtgcctgaagacctgcccagcctcccgggacctctgattggtgtgacttggtacacgtcaattcaatcaagaatgtaaggtggcataagaattatcatgtatgggagggtaaatgtagttctttccaatgatggaaaacggatgctgcttccgatgtaacaacatgtgattctttggactagaggtatactatttacagtaaagagtgaaaaaaaaggacttagctacatg

SEQ ID NO: 9 - protein, 320 aa, ARTIFICIAL SEQUENCE
  CHEMICALLY SYNTHESIZED
MARLVLSECCGLAPLRLRAGRRGAAIAAPSPAALSVAAAAVVPGRPASAAIHRDWALRVSAPTRLTSAVEEDKRSSPLGEEGVADSGAAGGDGFDPGAPPPFGLAEIRAAIPKHCWVKDPWRSMSYVLRDVVVVLGLAAAAARLDSWLVWPLYWAAQGTMFWALFVLGHDCGHGSFSNNPKLNSVVGHILHSSILVPYHGWRISHRTHHQNHGHVEKDESWHPLPERLYKSLDFMTKKLRFTMPFPLLAFPLYLFARSPGKTGSHFNPSSDLFQPNEKKDIITSTASWLAMVGVLAGLTFMMGPIPMLKLYGVPYLVFVA

SEQ ID NO: 10 - DNA, 3132 bp, ARTIFICIAL SEQUENCE
  CHEMICALLY SYNTHESIZED
cgcacacccaaaccccatcccgaacgcagaagatcccgtgcccgtgccagcggcggcgacggtgtgtgtggtgtggactggtagccatcatcaatccaggcatccccgtccacccgtaaccccgccgcccggcccccgcggacgccggcaatggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactggtacacctccacgattcccctgccgaactgtcctgttaagctttcaactcaggtctttgacttctgcttcatctctgctgtttgcagtggacacgggagcttctcaaacaaccccaagctcaacagcgtggtcggccacatactccattcctccattcttgtcccataccacggatggtaggattgcccatgtttcttacgcttgttactttgttagttagtttctgtcgaggattatcgttgatgttaaggaaggacctgtctttgaatggaattcaggaggattagccacaggacgcaccatcaaaaccacggccacgttgagaaggacgagtcctggcacccggtaggtttcctgtcgccctctcttcagttggagcttgagaagttatgctgatggtacatgtctatgaatggtgcttgatggttcctcggattgtgattacttgcagctaccagagaggctgtacaaaagcctggactttatgactaagaaattgcggttcaccatgccattccccctgctcgcgttcccgttatacttggtgaggctaagagcatctctattgtatgatacatgaattggacgaataaatgttttttgttttatggcgtggtagtggtgctaaattcctgccctgtgggttcatgattatgtagttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtaagagcgacatgtcctcctcttcagacccaacttctgcaaaaggataatttgaatgggtgatgtctgtgtgctcgaaatttatgtggctgctgctggtgtgtgcaggtatttgttgcttggctggatatggtcacatacttgcaccatcatggccacgaagacaagcttccctggtaccgtggaaaggtgattattagcaaagcaattttccaatttgttgatgtttcaaggtcccagtaatcttaaaaaactacagttacttccgtttttggttggttagaagcttttaaatgtttgcctcagctaagtcaggacgttattaattgcagcgaattgttgaatcataatacatcctatcctagaaatggaagttgtgtaaggactaaggacacaccatgccagaaatgttagatgcgcaatgacttcctctccttttgatttaccagtattttggataaaactggctgctttccattgcgccacttgacttcagcatctgtaggctatctgttcttgctggtagatacatgtttggtagactgatagacttcaacatctggactcactttgtttgcaaatggctagcttgaccttgaaacctttaaatcatatataaaccctagtggtggatattacaaaccaaagttctatccttggtaattttctggagtaagatctaattggaggatgctattgaactattggacaaaatgtctatttctcttttgtctttatgaggggaaatgatgtaactttgtatttcgtcgcttaccgagtgtaacgctaggtcagcctattatatagttttcagttttcactttacttttcagattatcgattgctgttaatttataagtattctatcctgatttctccttttgaagatagattggttattgataccacattgtccttttctatattaggaatggagttatctgcgtggaggactgacgacactcgaccgggactatggattgatcaacaatatccatcatgacattggaacccatgtcatccatcaccttttccctcaaatcccacattaccatctcattgaggcgataagagtttcccctattcttgcctttttttcatttttattataagtttctttgcctagatcataagacctgttcttggttgacctgcagactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattgacacggactatagaacgtagcgtattcttcatttttctcaggggggctgtgcctgaagacctgcccagcctcccgggacctctgattggtgtgacttggtacacgtcaattcaatcaagaatgtaaggtggcataagaattatcatgtatgggagggtaaatgtagttctttccaatgatggaaaacggatgctgcttccgatgtaacaacatgtgattctttggactagaggtatactatttacagtaaagagtgaaaaaaaaggacttagctacatg

SEQ ID NO: 11 - protein, 389 aa, ARTIFICIAL SEQUENCE
  CHEMICALLY SYNTHESIZED
MARLVLSECCGLAPLRLRAGRRGAAIAAPSPAALSVAAAAVVPGRPASAAIHRDWALRVSAPTRLTSAVEEDKRSSPLGEEGVADSGAAGGDGFDPGAPPPFGLAEIRAAIPKHCWVKDPWRSMSYVLRDVVVVLGLAAAAARLDSWLVWPLYWAAQGTMFWALFVLGHDCGHGSFSNNPKLNSVVGHILHSSILVPYHGWRISHRTHHQNHGHVEKDESWHPLPERLYKSLDFMTKKLRFTMPFPLLAFPLYLFARSPGKTGSHFNPSSDLFQPNEKKDIITSTASWLAMVGVLAGLTFMMGPIPMLKLYGVPYLVFVAWLDMVTYLHHHGHEDKLPWYRGKEWSYLRGGLTTLDRDYGLINNIHHDIGTHVIHHLFPQIPHYHLIEA

SEQ ID NO: 12 - DNA, 669 bp, ARTIFICIAL SEQUENCE
  CHEMICALLY SYNTHESIZED
atggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactgtggacacgggagcttctcaaacaaccccaagctcaacagcgtggtcggccacatactccattcctccattcttgtcccataccacggatggaggattagccacaggacgcaccatcaaaaccacggccacgttgagaaggacgagtcctggcacccg

SEQ ID NO: 13 - DNA, 1356 bp, ARTIFICIAL SEQUENCE
  CHEMICALLY SYNTHESIZED
atggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactgtggacacgggagcttctcaaacaaccccaagctcaacagcgtggtcggccacatactccattcctccattcttgtcccataccacggatggaggattagccacaggacgcaccatcaaaaccacggccacgttgagaaggacgagtcctggcacccgctaccagagaggctgtacaaaagcctggactttatgactaagaaattgtggttcaccatgccattccccctgctcgcgttcccgttatacttgttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtatttgttgcttggctggatatggtcacatacttgcaccatcatggccacgaagacaagcttccctggtaccgtggaaaggaatggagttatctgcgtggaggactgacgacactcgaccgggactatggattgatcaacaatatccatcatgacattggaacccatgtcatccatcaccttttccctcaaatcccacattaccatctcattgaggcgactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattga

SEQ ID NO: 14 - DNA, 1356 bp, ARTIFICIAL SEQUENCE
  CHEMICALLY SYNTHESIZED
atggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactgtggacacgggagcttctcaaacaaccccaagctcaacagcgtggtcggccacatactccattcctccattcttgtcccataccacggatggaggattagccacaggacgcaccatcaaaaccacggccacgttgagaaggacgagtcctggcacccgctaccagagaggctgtacaaaagcctggactttatgactaagaaattgcggttcaccatgccattccccctgctcgcgttcccgttatacttgttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtatttgttgcttagctggatatggtcacatacttgcaccatcatggccacgaagacaagcttccctggtaccgtggaaaggaatggagttatctgcgtggaggactgacgacactcgaccgggactatggattgatcaacaatatccatcatgacattggaacccatgtcatccatcaccttttccctcaaatcccacattaccatctcattgaggcgactgaggcagcaaaaccggttcttggcaagtactacaaagaaccaaagaagtcgggtgctcttccgtggcacctatttggggtgctagcacaaagcttaaagcaagaccactatgttagcgacaccggagatgtagtctactaccaaactgactcgaaaacgaacacctctgcacaaaaatctgattga

SEQ ID NO: 15 - DNA, 1167 bp, ARTIFICIAL SEQUENCE
  CHEMICALLY SYNTHESIZED
atggcgcggctcgtgctctccgagtgctgcggcctcgcgccgctgcgcctgcgcgccggccgccggggcgccgccattgcggcgccgtcgcccgccgcgctctccgtggcggcagcggcggtggtcccgggccgccccgcgtccgcggccatccaccgcgactgggcgctccgcgtctccgcgcccacccgcctcacctccgccgtcgaggaggacaagaggagctccccgctcggggaggagggcgtggcggacagcggcgccgcgggcggggacgggttcgacccgggggcgcccccgccgttcgggctggcggagatccgcgcggccatccccaagcactgctgggtcaaggacccctggcgctccatgagctacgtgctgcgtgacgtcgtcgtcgtcctgggactcgccgcggcggccgcgcgcctcgacagctggctcgtctggccgctctactgggccgcgcagggcaccatgttctgggcgctcttcgtccttggccacgactgtggacacgggagcttctcaaacaaccccaagctcaacagcgtggtcggccacatactccattcctccattcttgtcccataccacggatggaggattagccacaggacgcaccatcaaaaccacggccacgttgagaaggacgagtcctggcacccgctaccagagaggctgtacaaaagcctggactttatgactaagaaattgcggttcaccatgccattccccctgctcgcgttcccgttatacttgttcgcaaggagtccagggaagacaggatcacacttcaacccaagcagtgatttgttccaacctaatgaaaagaaggacatcataacatcaacggcctcctggctggccatggttggtgttctggctggtctcacctttatgatgggtcctattccgatgctaaagctctacggagtcccatacttggtatttgttgcttggctggatatggtcacatacttgcaccatcatggccacgaagacaagcttccctggtaccgtggaaaggaatggagttatctgcgtggaggactgacgacactcgaccgggactatggattgatcaacaatatccatcatgacattggaacccatgtcatccatcaccttttccctcaaatcccacattaccatctcattgaggcg

(19) United States
(12) Patent Application Publication
(10) Pub. No.: US 2019/0093120 A1
(43) Pub. Date: Mar. 28, 2019

(54) SORGHUM YIELD ENHANCEMENT GENE MULTI-SEEDED 3 (MSD3)

(71) Applicant: The United States of America, as represented by the Secretary of Agriculture, Washington, DC (US)

(72) Inventors: ZHANGUO XIN, LUBBOCK, TX (US); YINPING JIAO, LUBBOCK, TX (US); JUNPING CHEN, LUBBOCK, TX (US); GLORIA B. BUROW, LUBBOCK, TX (US); DOREEN WARE, MELVILLE, NY (US); JOHN J. BURKE, LUBBOCK, TX (US)

(21) Appl. No.: 15/712,372

(22) Filed: Sep. 22, 2017

(51) Int. Cl. C12N 15/82; C12N 9/02; C07K 14/415

(52) U.S. Cl. CPC C12N 15/8261; C12N 9/0071; C12N 15/827; C12N 15/8243; C07K 14/415; C12Y 114/19

(57) ABSTRACT

This invention relates to novel mutations in a sorghum gene, Msd3, which increase the seed yield in sorghum. The nucleic acid sequences of this mutated gene and its encoded protein(s) are included. This invention also relates to genetically altered plants having this mutated gene and/or containing the mutated protein and which have increased flower production and seed yield and lower jasmonic acid production.

### BACKGROUND OF THE INVENTION

### Field of Invention

This invention relates to novel mutations in a sorghum gene, Msd3, which increase the seed yield in sorghum. The nucleic acid sequences of this mutated gene and its encoded protein(s) are included. This invention also relates to genetically altered plants having this mutated gene and/or containing the mutated protein and which have increased flower production and seed yield and lower jasmonic acid production.

### Background

Seed number per panicle is a major determinant of grain yield in sorghum [Sorghum bicolor (L.) Moench] and other cereal crops (Ashikari et al., Science, (2005) 309:741-45; Boyles et al., The Plant Genome, (2016) 9: doi: 10.3835/plantgenome2015.09.0091; Duggan et al, Can. J. Plant Sci., (2000) 80:739-45; Reynolds et al, J. Exp. Bot., (2009) 60:1899-1918; Richards, R. A., J. Exp. Bot., (2000) 51:447-58; Saeed et al, Crop Sci., (1986) 26:346-51). Increased seed number and seed size, which is directly related to improved grain yield, is a common goal during domestication of cereal crops resulting in inadvertent selection of genetic stocks with greater seed number and larger seeds (Zohary et al, “Domestication of Plants in the Old World: The origin and Spread of Cultivated Plants in West Asia, Europe, and the Mediterranean Basin”, 4^{th} ed., (2012).

Seed number per panicle is determined by several attributes of the inflorescence, including the number, and length, of the primary and secondary flower branches, and fertility of spikelets. In sorghum, and in the Panicoideae, the inflorescence or panicle consists of a main rachis on which many primary branches are developed. Secondary branches, sometimes, tertiary branches are developed from the primary branches (Brown et al, Theor. Appl. Gen., (2006) 113:931-42; Burow et al, Crop Sci., (2014) 54:2030-37). The main inflorescence, primary branches, secondary, and tertiary branches, all end with a terminal triplet of spikelets, which consists of one sessile bisexual spikelet and two lateral staminate pedicellate spikelets (Walters & Keil, Vascular Plant Taxonomy, 4^{th} ed., 1988). Below the terminal spikelets, one or more spikelet pair can develop, and these adjacent spikelet pairs consist of one sessile and one pedicellate spikelet. In the wild type sorghum, BTx623, and all other characterized natural sorghum accessions, only the sessile spikelets are perfect flowers and can develop into seeds. The development of pedicellate spikelets is arrested at various stages in different sorghum lines. In some lines, the pedicellate spikelets can develop anthers and shed viable pollen, but few lines can develop ovary and produce viable seeds (Karper & Stephens, J. Hered., (1936) 27:183-94). Thus, the pedicellate spikelets in the wild type eventually abort.

Recently, a series of sorghum mutants that bear both fertile sessile and pedicellate spikelets was isolated and characterized. They were termed multiseeded (msd) mutants because their panicles are capable of producing three times the seed numbers as compared to the non-mutated BTx623 (Burow et al, supra). Previously, the Msd1 gene was identified as a TCP-domain plant-specific transcription factor through next-generation sequencing of the pooled genomic DNA of homozygous mutants selected from a backcrossed F2 population derived from a cross of msd1-1 (p12) to BTx623 and Msd2 gene through direct sequencing of allelic msd2 mutants.

As described herein, we describe the identification of the Msd3 gene and the production of msd3 mutant plants and seeds. This new class of msd mutants contains no mutation in Msd1 or Msd2 genes. The Msd3 gene encodes plastid-targeted co-3 fatty acid desaturase, which catalyzes the desaturation of linoleic acid (18:2, 18 carbon-chain with two double bonds) to linolenic acid (18:3, 18 carbon chain with three double bonds). FIG. 1 illustrates the biosynthetic pathways of jasmonic acid (JA) from linoleic acid (Stintzi & Browse, Proc Nat'l Acad. Sci. USA, (2000) 97:10625-30; Wasternack & Hause, Annals Bot., (2013) 8:26). Galactolipids, including monogalactosyldiacylglycerol (MGDG) and digalactosyldiacylglycerol (DGDG), from the chloroplast membranes are cleaved off by a lipase to release free linolenic acid, which serves as the substrate for JA biosynthesis (Wasternack & Hause, supra). The MSD2 protein (13-lipoogenase III) catalyzes the conversion of free linolenic acid to 13-hydroperoxylenolenic acid, which is the first committed step for JA biosynthesis.

### SUMMARY OF THE INVENTION

Provided herein, in one embodiment of the invention is a genetically altered plant or parts thereof that has a mutation in a gene encoding a plastid-targeted ω-3 fatty acid desaturase having a nucleic acid sequence at least 85% identical to SEQ ID NO: 4, SEQ ID NO: 6, SEQ ID NO: 8, SEQ ID NO: 10, SEQ ID NO: 12, SEQ ID NO: 13, SEQ ID NO: 14, or SEQ ID NO: 15, and where the mutation causes a reduction in the enzymatic activity of the plastid-targeted omega-3 fatty acid desaturase compared to the enzymatic activity of a wild-type plant. In some embodiments, the genetically altered plant has reduced amount of 8-cis-jasmonic acid in its leaf tissue compared to the amount of 8-cis-jasmonic acid in leaf tissue of a non-genetically altered plant. In particular embodiments, the plant is a cereal crop plant such as sorghum, maize, rice, barley, oats, wheat, rye, millet, and triticale. In preferred embodiments, the mutation is present in two copies in the genetically altered plant. In some embodiments, the plant part is a plant cell, a seed, or pollen.

Another embodiment of the invention provided herein is a genetically altered plant or parts thereof, that has a null mutation in at least two copies of a gene encoding a plastid-targeted ω-3 fatty acid desaturase having a nucleic acid sequence that encodes a protein at least 80% identical to SEQ ID NO: 2. In some embodiments, the genetically altered plant has reduced amount of 8-cis-jasmonic acid in its leaf tissue compared to the amount of 8-cis-jasmonic acid in leaf tissue of a non-genetically altered plant. In particular embodiments, the plant is a cereal crop plant such as sorghum, maize, rice, barley, oats, wheat, rye, millet, and triticale. In preferred embodiments, the mutation is present in two copies in the genetically altered plant. In some embodiments, the plant part is a plant cell, a seed, or pollen.

Further provided herein is an aspect of the invention that is a method for constructing a genetically altered cereal crop that has the MSD3 phenotype, where the methodology includes the steps of: (i) introducing an altered msd3 nucleic acid into a cereal crop or grass plant to provide a genetically altered cereal crop or grass plant; and (ii) selecting the genetically altered cereal crop that is homozygous for the altered msd3 nucleic acid, thereby constructing the genetically altered cereal crop or grass plant, wherein the genetically altered cereal crop or grass plant has the MSD3 phenotype. In particular embodiments, the cereal crop is selected from the group consisting of sorghum, maize, rice, barley, oats, wheat, rye, millet, and triticale. In a specific embodiment, the cereal crop is sorghum and the altered msd3 nucleic acid is selected from the group consisting of SEQ ID NO: 4, SEQ ID NO: 6, SEQ ID NO: 8, SEQ ID NO: 10, SEQ ID NO: 12, SEQ ID NO: 13, SEQ ID NO: 14, or SEQ ID NO: 15. Such methodology can be achieved via any method capable of introducing the altered msd2 nucleic acid into the plant or plant part, including introgression, genomic editing, or exposing said cereal crop or grass plant to a mutagen, and said selecting said genetically altered cereal crop or grass plant occurs via marker assisted selection. In preferred embodiments, the MSD3 phenotype arises from a mutation in a plastid-targeted co-3 fatty acid desaturase and where the genetically altered cereal crop has a reduced amount of 8-cis-jasmonic acid in its leaf tissue compared to the amount of 8-cis-jasmonic acid in leaf tissue of a non-genetically altered plant.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides a graphic representation of jasmonic acid biosynthesis from linolenic acid. The Msd3 gene encodes a chloroplast-targeted co-3 desaturase that catalyzes the conversion of linoleic acid (18:2) moiety on galactolipids to linolenic acid (18:3). A lipase, still unknown in sorghum, cleaves the linolenic acid off the galactolipids. The free linolenic acid is converted to 13-hydroperoxyoctadeca-9,11,15-trienoic acid by Msd2, a class II 13-lipoxygenase, to initiate JA biosynthesis. (Adapted from Lyons at al, Plant Cell Rep., (2013) 32:815-27).

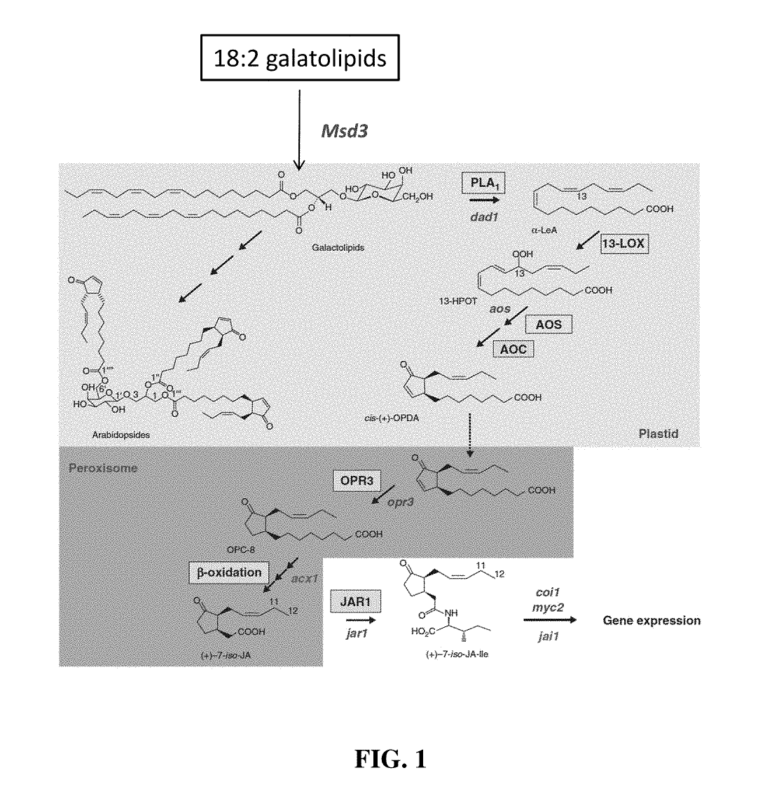

FIG. 2 provides a graphic representation of the identification of the Msd3 gene by MutMap. Four F2 populations, BTx623*p6, BTx623*p14, BTx623*p21, and BTx623*p24, were subjected to sequencing, each with 20 bulked F2 mutants. Only one gene (Sobic.001G407600 or Msd3) is commonly mutated in all four populations. In the p21 and p24 populations, the Msd3 gene was mutated at the same position.

FIG. 3 provides a pictorial representation of the Msd3 gene. The Msd3 gene, Sobic.001G407600, spans a genomic sequence of 3132 bp. The coding sequence (SEQ ID NO: 2) is 1356 bp and encodes a predicted protein of 451 amino acids (SEQ ID NO: 3). The gene has 8 exons (boxes) and 7 introns (horizontal line). The vertical lines indicate mutation sites as described in more detail herein.

FIG. 4 provides a graphic representation of co-segregation of the msd3-4 mutation with the msd panicle phenotype. The mutation identified from msd3-4 (FIG. 3) was converted to a KASP marker. The AA indicated that the F2 plants have the homozygous mutation genotype of AA. The GA indicated that the F2 plants carried the heterozygous gene type. The GG indicated that the F2 plants carry the unmutated genotype at the msd3-4 site. All F2 plants carrying AA displayed the msd panicle.

FIG. 5 provides graphic representation of lipid profiles of leaves and panicles from PTx623 and msd3-3 (SBp6) mutant. Lipid profiles in the youngest mature leaves and panicles were carried out by the Lipidomics Center at Kansas State University. The number in parenthesis indicates the length and number of double bonds of the fatty acid moiety. (36:6) represents lipid species with 2 linolenic acid (18:3); (36:4) represents lipid species with 2 linoleic acid (18:2); and (36:5) represents lipid species with 1 linolenic acid and 1 linoleic acid moiety. Minor lipids less than 1% molar percentage were not plotted.

FIG. 6 provides graphic representation of jasmonic acid (JA) levels in the leaves of PTx623 and msd3 mutants. The levels of jasmonic acid in leaves and panicles of BTx623 and msd3 mutants were determined by the Chemistry Research Unit of USDA-ARS at Gainesville, Fla. The cis-JA levels, the biologically active form, were reduced in all three msd3 mutants in comparison with BTx623. JA levels in panicles were below the detection limit.

### STATEMENT OF DEPOSIT

On or before May 23, 2017, the inventors deposited 2,500 seed of Sorghum bicolor strain msd3-1 (p24), as described herein, with American Type Culture Collection (ATCC) located at 10801 University Blvd., Manassas, Va. 20110, in a manner affording permanence of the deposit and ready accessibility thereto by the public if a patent is granted. The deposit has been made under the terms of the Budapest Treaty on the International Recognition of the Deposit of Microorganisms for the Purposes of Patent Procedure and the regulations thereunder. The deposit's accession number is ATCC Accession Number PTA-124206. All restrictions on the availability to the public of S. bicolor strain msd3-1 (p24) which has been deposited as described herein will be irrevocably removed upon the granting of a patent covering this particular biological material. The S. bicolor strain msd3-1 (p24) has been deposited under conditions such that access to the organism is available during the pendency of the patent application to one determined by the Commissioner to be entitled thereto under 37 C.F.R. § 1.14 and 35 U.S.C § 122.

For the purposes of this invention, any S. bicolor plants or portions thereof, seeds or progeny thereof, having the identifying characteristics of PTA-124206, including variants thereof which have the identifying characteristics and activity as described herein are included.

The deposited biological material will be maintained with all the care necessary to keep it viable and uncontaminated for a period of at least five years after the most recent request for the furnishing of a sample of the deposited microorganism, and in any case, for a period of at least thirty (30) years after the date of deposit for the enforceable life of the patent, whichever period is longer.

### DETAILED DESCRIPTION OF THE INVENTION

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Genetically altered sorghum plants examined herein have a “multi-seeded” mutation #3 (MSD3) with a distinctive phenotype and genetic alterations that differs from previously identified genetically altered MSD1 and MSD2 sorghum. The genetically altered sorghum plants described herein have at least one mutation in a gene referred to herein as MSD3. The equivalent gene in sorghum strain BTx623 is identified as Sobic.001G407600 (SEQ ID NO: 1, the wild-type genomic sequence and SEQ ID NO: 2, the wild-type coding sequence). Furthermore, Sobic.001G407600 (MSD3) shows high identity to other chloroplast-targeted linoleic acid desaturases from rice Fad7 (OsFAD7_LOC_Os03g18070.1) and Fad8 (OsFAD8_LOC_Os07g49310.1), as well as corn Fad7 (ZmFAD7_Zm00001d047743_P001) and Fad8 (ZmFAD8_Zm00001d028742_P001).

This invention involves MSD3 genetic alterations in sorghum and other cereal crops, including but not limited to, corn/maize, rice, barley, oats, wheat, rye, millet, and triticale, which leads to an increase in the number of seeds produced per plant which is an important component to an increase in the plant's grain yield. These MSD3 genetic alterations result in the MSD3 phenotype for which the genetically altered plant has an increased number of flower branches, increased size of flower branches, full fertility of pedicellate spikelets, increased number of flowers, and increased number of seeds (grain). Genetically altered cereal crops having an MSD3 genetic alteration have reduced or no activity of the chloroplast-targeted co-3 desaturase, catalyzing the desaturation of linoleic acid (18:2) to linolenic acid (18:3) encoded by the MSD3 gene. Further, the genetically altered cereal crops of the present invention have highly reduced levels of 8-cis-jasmonic acid production is reduced or completely inhibited in developing panicles and leaves in the genetically altered cereal crop plants having the MSD3 phenotype.

The invention described herein covers any monocot plant (such as cereal crops and grasses) which contains a genetic alteration in a chloroplast-targeted linoleic acid desaturase that has high identity, at the amino acid level, to sorghum's Sobic.001G407600. Included are the particular mutations described herein and other genetic alterations resulting in reduced or no activity of the encoded enzyme compared to the wild-type plant's enzymatic activity. The genetic alteration could be a single point mutation in the DNA at the specific nucleotides discussed below, a deletion mutation in the DNA, a null mutation, or another mutation which reduces or inhibits the enzymatic activity of the encoded enzyme. A plant with this MSD3 mutation would have more flower production and more seed production compared to the wild-type plant. The mutation could be a change in the DNA sequence of the gene resulting in (1) early termination of mRNA translation into a protein, (2) introduction of a stop codon resulting in truncation of the wild-type protein, (3) altering the enzymatic activity of the encoded protein by changing one or more amino acids to one or more different amino acids, or (4) altering the enzymatic activity of the encoded protein by abolishing a splice site between an exon and an intron or an intron and an exon. These types of mutations cause reduced or no enzymatic activity of the encoded protein which results in the observed phenotype. Further any introduced null mutation will result in this MSD3 phenotype. In particular embodiments, genetically altered plants of the present invention have one or more of the altered genomic or coding sequences set forth in SEQ ID NO: 4 (p24/p21 genomic sequence; msd3-1), SEQ ID NO: 6 (p14 genomic sequence; msd3-2), SEQ ID NO: 8 (p6 genomic sequence; msd3-3), SEQ ID NO: 10 (P37 genomic sequence; msd3-4), SEQ ID NO: 12 (p24/p21 predicted coding sequence), SEQ ID NO: 13 (p14 predicted coding sequence), SEQ ID NO: 14 (p6 predicted coding sequence), and SEQ ID NO: 15 (p37 predicted coding sequence).

In some embodiments of the present invention, genetically altered sorghum is provided for which the genetic alteration results in a reduction in the amount of jasmonic acid (8-cis jasmonic acid) in the genetically altered sorghum's panicle tissue compared to the amount of jasmonic acid in a non-genetically altered sorghum's panicle tissue. In particular embodiments, genetically altered sorghum of the present invention produces an altered MSD3 protein having the amino acid sequence set forth in SEQ ID NO: 5 (msd3-1; p24/p21), SEQ ID NO: 7 (msd3-2; p14), SEQ ID NO: 9 (msd3-3; p6), SEQ ID NO: 11 (msd3-4; p37), or no MSD3 protein. The altered MSD3 protein is generated from a DNA coding sequence (“coding sequence”) set forth in either SEQ ID NO: 4 (msd3-1; p24/p21), SEQ ID NO: 6 (msd3-2; p14), SEQ ID NO: 8 (msd3-3; p6), or SEQ ID NO: 10 (msd3-4; p37), or a sequence that results in the production of such proteins and proteins with at least 80%, 81%, 82%, 83%, 84%, 85%, 86%, 87%, 88%, 89%, 90%, 91%, 92%, 93%, 94%, 95%, 96%, 97%, 98%, or 99% identity to any of these.

In another embodiment, this invention involves genetically altered plants (monocots in one embodiment; cereal crops in another embodiment; grasses in a third embodiment) that have an altered MSD3 gene which results in a reduction in the amount of jasmonic acid in the panicle or leaf tissue compared to the amount of jasmonic acid in panicle or leaf tissue in non-genetically altered plants. In one embodiment, the genetically altered plant produces an altered MSD3 protein having the amino acid sequence that is at least 80% identical to the amino acid sequence set forth in either SEQ ID NO: 5, SEQ ID NO: 7, SEQ ID NO: 9 or SEQ ID NO: 11. In this embodiment, the altered MSD3 protein is generated from a coding sequence that encodes this altered MSD3 protein. One of skill in the art will recognize that, because the MSD3 phenotype is recessive, all functional wild-type alleles must be replaced with non-functional alleles. Non-functional alleles can be the same allele, or different alleles. In another embodiment, any genetically altered plant that contains a null mutation in this chloroplast-targeted linoleic acid desaturase gene is included in this invention. For this invention, a null mutation is a DNA alteration that results in no production of the chloroplast-targeted linoleic acid desaturase protein, or in production of a protein that lacks chloroplast-targeted linoleic acid desaturase activity.

A mutation to a splice site sequence (a splice site mutation) which causes incorrect splicing of mRNA can be a null mutation. A splice site mutation is a mutation that inserts, deletes or changes a number of nucleotides in the specific site at which splicing takes place during the processing of precursor mRNA into mature mRNA. Splice site consensus sequences that drive exon recognition are located at the very termini of introns. The deletion of the splicing site results in one or more introns remaining in mature mRNA and may lead to the production of abnormal proteins. When a splice site mutation occurs, the mRNA transcript possesses information from these introns that normally should not be included. Introns are supposed to be removed, while the exons are expressed. The mutation must occur at the specific site at which intron splicing occurs: within non-coding sites in a gene, directly next to the location of the exon. The mutation can be an insertion, deletion, frame shift, etc. The splicing process itself is controlled by the given sequences, known as splice-donor and splice-acceptor sequences, which surround each exon. Mutations in these sequences may lead to retention of large segments of intronic DNA by the mRNA, or to entire exons being spliced out of the mRNA. These changes could result in production of a nonfunctional protein. An intron is separated from its exon by means of the splice site. Acceptor-site and donor-site relating to the splice sites signal to the splicesome where the actual cut should be made. These donor sites, or recognition sites, are essential in the processing of mRNA.

Plants, parts of plants, and progeny that “exhibit” or “have” the MSD3 phenotype have the genetic alteration involving a mutation in the gene encoding chloroplast-targeted linoleic acid desaturase that has high identity, at the amino acid level, to sorghum's Sobic.001G407600 (MSD3), or homologs from other plants and which have reduced levels or complete inhibition of jasmonic acid production in developing panicle. However, a part of a plant, such as but not limited to, a cell, a protocorm, a pollen, a seed, etc., does not have a panicle. Thus, one is unable to determine if the part of the plant “exhibits” or “has” the MSD3 phenotype simply by looking at the plant's part. However, if the part of the plant has the genetic alteration that is described herein or which can be determined using the molecular biology techniques described herein, then that plant part is considered to “exhibit” or “have” the MSD3 phenotype. A plant that lacks a genetic alteration of the gene encoding the chloroplast-targeted linoleic acid desaturase (Sobic.001G407600, or an otholog) that does not result in the MSD3 phenotype, and thus the plant does not exhibit the MSD3 phenotype, is referred to as a “wild-type” or “non-genetically altered” plant, even if the plant has a genetic alteration in the chloroplast-targeted linoleic acid desaturase (Sobic.001G407600, or an otholog) that does not give rise to the MSD3 phenotype or has a genetic alteration in one or more other genes. Such a wild-type plant is a plant that fails to exhibit the MSD3 phenotype.

It is noted that the MSD3 phenotype is a recessive phenotype. A genetically altered plant needs two copies of the mutated gene to exhibit the MSD3 phenotype, or any other genetic background resulting in the absence of a functional Msd3 protein. Further, the mutated gene is passed to offspring in a simple Mendelian genetic pattern. The altered alleles need not be the same allele, as long as the combination of genetically altered alleles results in the MSD3 phenotype.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The term “about” is defined as plus or minus ten percent of a recited value. For example, about 1.0 g means 0.9 g to 1.1 g and all values within that range, whether specifically stated or not.

The terms “identical” or percent “identity”, in the context of two or more nucleic acids or polypeptide sequences, refer to two or more sequences or subsequences that are the same or have a specified percentage of amino acid residues or nucleotides that are the same (e.g., 80%, 85% identity, 90% identity, 99%, or 100% identity), when compared and aligned for maximum correspondence over a designated region as measured using a sequence comparison algorithm or by manual alignment and visual inspection.

The phrase “high percent identical” or “high percent identity”, in the context of two polynucleotides or polypeptides, refers to two or more sequences or subsequences that have at least about 80%, identity, at least about 80%, 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid residue identity, when compared and aligned for maximum correspondence, as measured using a sequence comparison algorithm or by visual inspection. In an exemplary embodiment, a high percent identity exists over a region of the sequences that is at least about 50 residues in length. In another exemplary embodiment, a high percent identity exists over a region of the sequences that is at least about 100 residues in length. In still another exemplary embodiment, a high percent identity exists over a region of the sequences that is at least about 150 residues or more in length. In one exemplary embodiment, the sequences are high percent identical over the entire length of the nucleic acid or protein sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al. (eds.), Current Protocols in Molecular Biology, 1995 supplement).

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants). It includes plants of a variety of ploidy levels, including aneuploid, polyploid, diploid, haploid and hemizygous. The genetically altered plants described herein can be monocot crops such as, but not limited to, sorghum, maize, wheat, rice, barley, oats, rye, millet, and triticale.

A genetically altered organism is any organism with any changes to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has mutations in its DNA caused by the one or more mutagens, as compared to the wild-type organism (i.e, organism not subjected to the mutagens). Also, an organism that has been bred to incorporate a mutation into its genetic material is a genetically altered organism. For the purposes of this invention, the organism is a plant.

Once a genetically altered plant has been generated, one can breed it with a wild-type plant and screen for heterozygous F1 generation plants containing the genetic change present in the parent genetically altered plant. Then F2 generation plants can be generated which are homozygous for the mutation. These heterozygous F1 generation plants and homozygous F2 plants, progeny of the original genetically altered plant, are considered genetically altered plants, having the altered genomic material from a parent plant that has been genetically altered.

As discussed briefly above, one can subject a plant's seeds to a mutagen, then grow the seeds, and screen the plants for altered phenotypes. The plants with altered phenotypes will have one or more mutations within the plant's DNA (either within the organelles or nucleus) that cause the altered phenotype. Such genetically altered plants can then be bred as described above to generate homozygous genetically altered plants.

Another way to create mutations in sorghum's Sobic.001G407600 gene, or homologs and orthologs from other monocot plants is through genomic editing. Recombinant DNA restriction enzymes can be engineered by fusing a nuclease, for example FokI, with a structure that binds to a site in the MSD3 homologs, as specified by zinc finger, TALEN (transcription activator-like effector nuclease), or by CRISPR (clustered regularly interspaced short palindromic repeat)-Cas9 system, to make a double strand cut within the MSD3 homolog and replace with an engineered nucleic acids identified from the msd3 mutants. FokI is a bacterial type IIS restriction endonuclease consisting of an N-terminal DNA-binding domain, which can be made to bind specific DNA sequences in genome and a non-specific DNA cleavage domain at the C-terminal. See, Belhaj, et al., Plant Methods 9(1):39 (2013); Nekrasov, et al., Nat. Biotechnol. 31:691-693 (2013); Voytas, D. F., Annu. Rev. Plant Biol. 64:327-350 (2013); Shan, et al., Nat. Biotech. 31:686-688 (2013); and Li, et al., Methods 69(1):9-16 (2014). Genetically altered plants having mutations in Sobic.001G407600, or an otholog or homolog can be selected using any relevant methodology known in the art.

One such selection methodology is marker-assisted selection, a method of selecting desirable individuals in a breeding scheme based on DNA molecular marker patterns instead of, or in addition to, their phenotypic traits. Marker-assisted selection provides a useful tool that allows for efficient selection of desirable crop traits and is well known in the art (see, e.g., Podlich, et al., Crop Sci. 44:1560-1571 (2004); Ribaut and Hoisington, Trends in Plant Science 3:236-238 (1998); Knapp, S., Crop Science 38:1164-1174 (1998); Hospital, F., Marker-assisted breeding, pp 30-59, in Plant molecular breeding, H. J. Newbury (ed.), Blackwell Publishing and CRC Press (Oxford and Boca Raton).

As is well known in the art, breeders typically improve crops by crossing plants with desired traits, such as high yield or disease resistance, and selecting the best offspring over multiple generations of testing. Thus, new varieties can easily take eight to ten years to develop. In contrast to conventional selection methods, with marker-assisted selection plants are selected based on molecular marker patterns known to be associated with the traits of interest. Thus, marker-assisted selection involves selecting individuals based on their marker pattern (genotype) rather than their observable traits (phenotype). Thus, molecular marker technology offers the possibility to speed up the selection process and thus offers the potential to develop new cultivars quickly.

Therefore, in an exemplary embodiment, marker assisted selection is used to develop new cereal crops and/or grasses, and, in particular, sorghum cultivars, having the MSD3 phenotype. In this embodiment, the single nucleotide polymorphisms disclosed herein are used as markers to select for the MSD3 phenotype.

After one obtains a genetically altered plant expressing the MSD3 phenotype, one can efficiently breed the genetically altered plant with other plants containing desired traits. One can use molecular markers (i.e., polynucleotide probes) based on the SNP of msd3 gene to determine which offspring of crosses between the genetically altered plant and the other plant have the polynucleotide encoding msd3. This process is known as Marker Assisted Rapid Trait Introgression (MARTI). Briefly, MARTI involves (1) crossing the genetically altered MSD3 plant with a plant line having desired phenotype/genotype (“elite parent”) for introgression to obtain F1 offspring. The F1 generation is heterozygous for msd3 gene. (2) Next, an F1 plant is be backcrossed to the elite parent, producing F1 plants which genetically produces 50% wild-type and 50% heterozygote msd3 plants. (3) PCR using polynucleotide probes is performed to select the heterozygote genetically altered plants containing msd3 gene. (4) Selected heterozygotes are then backcrossed to the elite parent to perform further introgression. (5) This process of MARTI is performed for another four cycles. (6) Next, the heterozygote genetically altered plant is self-pollinated by bagging to produce the F2 generation. The F2 generation produces a phenotypic segregation ratio of 3 wild-type parent plants to 1 genetically altered MSD3 plant. (7) One selects genetically altered MSD3 plants at the F2 generation at the seedling stage using PCR with the polynucleotide probes and can optionally be combined with phenotypic selection at maturity. These cycles of crossing and selection can be achieved in a span of 2 to 2.5 years (depending on the plant), as compared to many more years for conventional backcrossing introgression method. Thus, the application of MARTI using PCR with polynucleotide probes significantly reduces the time to introgress the MSD3 genetic alteration into elite lines for producing commercial hybrids. The final product is an inbred plant line almost identical (99%) to the original elite in-bred parent plant that is the homozygous for msd3 gene.

Having generally described this invention, the same will be better understood by reference to certain specific examples, which are included herein to further illustrate the invention and are not intended to limit the scope of the invention as defined by the claims.

### EXAMPLES

### Example 1

MSD3 Mutant Production, Screening and Breeding

The sorghum msd3 mutants were identified from a pedigreed sorghum mutant library that was created by mutagenizing S. bicolor BTx623 seeds with the chemical mutagen, ethyl methane sulfonate (EMS) (Xin et al., BMC Plant Biol., (2008) 8:103). The wild type BTx623 and the msd mutants and their backcrossed F2 populations were planted in the field of USDA-ARS Cropping Systems Research Laboratory at Lubbock, Tex. (33′39″ N, 101′49″ W). During late grain filling stage, when the msd phenotype could be easily observed, leaf samples were collected from each of the confirmed homozygous msd mutants to prepare genomic DNA using a method described earlier (Xin & Chen, Plant Methods (2012) 8:26).

Homozygous msd3 mutants were identified from the backcrossed F2 populations for SBp6, SBp14, and SPp21/SBp24 during grain filling stage during which the msd phenotype could be easily scored. Genomic DNA pooled from the homozygous msd mutants from each F2 population was sequenced to >20× coverage on Illumina HiSeq2000. Low quality reads, adaptor sequences, and contamination were first excluded from the raw reads. Then the clean reads were aligned to the sorghum reference genome v3.0 with Bowtie2 (Langmead & Salzberg, Nat. Meth., (2012) 32:815-27). The SNP calling was done by Samtools and Bcftools using only the reads with mapping and sequencing quality more than 20 (Li et al., Bioinformatics, (2009) 25:2069-79). The read depth for true SNPs was set from 3 to 50. Because EMS is known to induce only G/C to A/T transition mutations (Greene et al., Genetics, (2003) 164:731-40), only the homozygous and G/C to A/T SNPs were used to predict the effect on gene function by Ensembl variation predictor (McLaren et al., Bioinformatics, (2010) 26: 2069-70). The homology analysis and functional annotation of candidate genes were obtained from Gramene database release 39 (Monaco et al., Nucl. Acids Res., (2014) 42:D1193-99) (www.gramene.org).

To confirm the causal SNPs discovered through the above sequencing analysis, the method known as Kompetitive Allele Specific PCR (KASP) by design (KBioscienc/LGC Genomics (www.lgcgenomics.com)) were used to confirm the SNPs according to the manufacturer's protocols with some modifications. About 200 bp of genomic DNA sequence spanning the causal SNP was submitted to KBioscience to design the allele specific primers. The marker amplification and analysis were conducted in-house at the Plant Stress and Germplasm Development Unit at Lubbock, Tex. Briefly, touchdown PCR from 65° C. to 57° C. was used for each pair of primers of the SNPs. After the touchdown step, the amplification was continued for 30 cycles using an annealing temperature of 57° C.

Phenotype of the Msd3 Mutants

From the pedigreed sorghum mutant library created at the Plant Stress and Germplasm Development Research Unit of USDA-ARS at Lubbock, Tex., we identified a series of mutants that have coordinated changes of increased number of flower branches, increased size of flower branches, and full fertility of pedicellate spikelets (Burow et al., supra; Xin et al., supra). These coordinated phenotypic changes lead to a potential of three-fold increase in seed number and two-fold increase in seed weight per panicle. In all characterized natural sorghum lines, only the sessile spikelets are fertile. Like other characterized msd mutants, msd3 mutants have increased panicle size and both sessile and pedicellate spikelets produced complete flowers and set seeds. We noticed that msd3 appeared to have larger seeds in comparison with msd1 and msd2 mutants (Table 1). Three replicates consisting of 100 seeds were weighed from each line. Seed weight was the average of the three measurements plus and minus the standard deviation.). The large seed size may make msd3 especially useful in improving grain yield and overall application in breeding programs.

TABLE 1
Seed size (mg/seed) of wild-type, msd1,
msd2 and msd3 mutants.

| Line | Seed Weight (mg) |
|---|---|
| BTx623 | 29.8 ± 0.32 |
| SBp12 (msd1-1) | 18.4 ± 0.36 |
| SBp15 (msd1-5) | 15.6 ± 0.45 |
| SBp4 (msd2-1) | 14.4 ± 0.91 |
| SBp8 (msd2-2) | 15.5 ± 0.23 |
| SBp6 (msd3-3) | 23 ± 0.39 |
| SBp14 (msd3-2) | 20.9 ± 0.26 |
| SBp24 (msd3-1) | 23.6 ± 0.93 |

Identification of Candidate Genes from Three Independent Alleles of Msd3

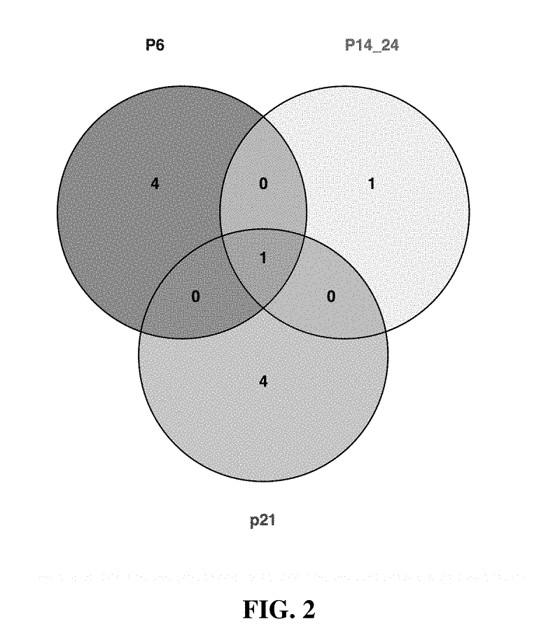

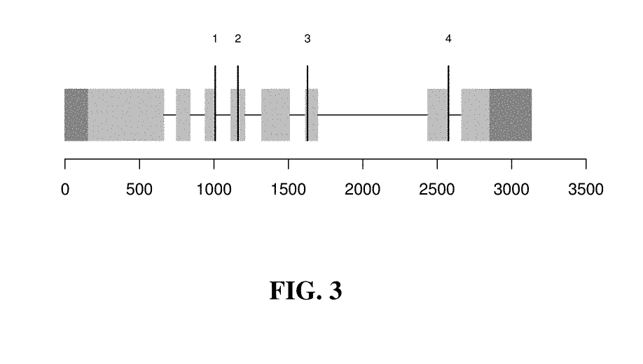

The Msd3 gene was identified through sequencing 20 bulked F₂ msd mutants selected from a segregating F₂ populations using an in-house bioinformatic pipeline similar to MutMap as described in rice (Abe et al., Nat. Biotechnol., (2012) 30:174-8). As described, we have crossed SBp6 (msd3-3), SBp14 (msd3-2), SBp21 (msd3-1), and SBp24 (msd3-1) to BTx623 and derived 4 F₂ populations. After bioinformatics analysis of the four bulked F₂ pools, we identified only one gene (Sobic.001G407600, Msd3) that carried homozygous mutations in all four bulked F₂ pools (FIG. 2). The genomic sequence of Msd3 is 3132 bp (SEQ ID No: 1) with a CDS of 1356 (SEQ ID No: 2), which encodes a protein with 451 amino acid (SEQ ID No: 3). The SBp21 and SBp24, renamed msd3-1, harbored the same mutation that converted the G residue at Chr01_6916308 to A, creating a splice site mutation at the junction of the third exon and the third intron (FIG. 3, SEQ ID NO: 4, SEQ ID NO: 5). SBp14, renamed as msd3-2, harbored a mutation of G to A transition at Chr01-69163762, which created a nonsynonymous mutation, R240W, on the MSD3 protein (FIG. 3, SEQ ID NO: 6, SEQ ID NO: 7). SBp6, renamed as msd3-3, harbored a transition mutation of G to A at Chr01_69164229, resulting the premature stop codon (W321*) in MSD3 protein (FIG. 3, SEQ ID NO: 8, SEQ ID NO: 9). Afterward, we identified another mutation in the Msd3 gene from sequenced mutant library (ASR106, 25M2-1370) at Chr_69165175, which also created a splice site mutation at the junction of 7^{th} exon and 7^{th} intron (FIG. 3, SEQ ID NO: 10, SEQ ID NO: 11). We named this msd mutant SBp37 and msd3-4. The interrupted CDS sequences for all msd3 mutants are provided as listed in SEQ ID NO: 12, SEQ ID NO: 13, SEQ ID NO: 14, and SEQ ID NO: 15.

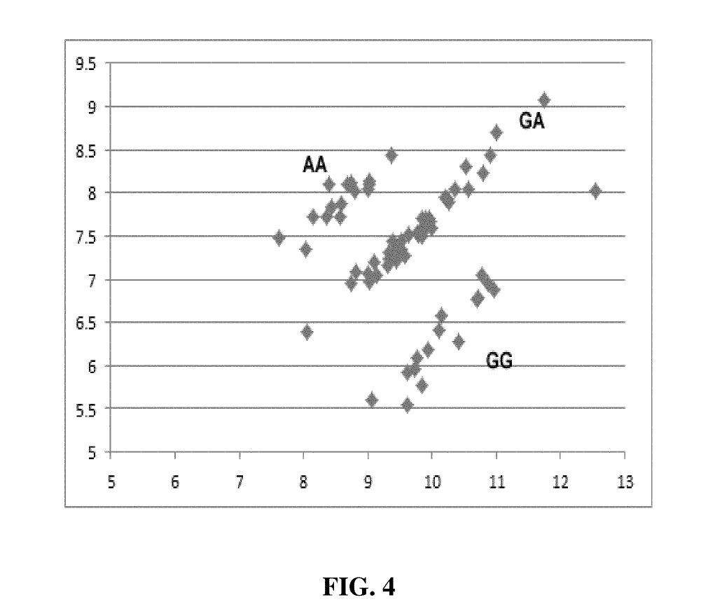

To determine if these mutations in Msd3 genes resulted in the msd phenotype, we made pairwise crosses among these four msd3 mutants. All Fᵢ plants from pairwise crosses displayed the msd phenotype. Thus, the evidence for the gene Sobic.001G407600 to be Msd3 is very strong. Furthermore, we tested the co-segregation of the msd3 mutation in SBp37 with the msd phenotype with KASP (FIG. 4). Among 63 individuals F₂ plants derived from a cross of BTx623*msd3-4 (SBp37), 15 plants were scored as AA at the mutation site. All 15 lines displayed the expected msd3 phenotype. Fifteen plants were scored as GG and the 33 scored as G/A. All these two classes displayed the wild-type panicle, indicating that msd3 is a recessive mutation and the mutation segregated perfectly with the msd phenotype.

Msd3 Encodes a Chloroplast-Targeted ω-3 Fatty Acid Desaturase

Sequencing analysis indicates that the Msd3 gene (Sobic.001G407600) encodes a chloroplast targeted fatty acid desaturase that adds a double bond to the co-3 carbon of linoleic acid (18:2) and converts it to linolenic acid (18:3) in the chloroplast. The gene has been annotated as fatty acid desaturase 8 (Fad8) according to Gramene (ensembl.gramene.org/Multi/Search/Results?species=all;idx=;q=Sobic.001 G407600;site=ensem blunit).

Most plants, including sorghum, have two closely related chloroplast-targeted linoleic (18:2) desaturase, termed Fad7 and Fad8 (Berberich et al., Plant Mol. Biol., (1998) 36:297-306; Li et al., The Plant Cell Online, (2003) 15:1646-61; Roman et al., Mol. Plant, (2015) 8:1599-1611; Tovuu et al., Plant Physiol. Biochem., (2016) 109:525-535). The other predicted chloroplast-targeted co-3 fatty acid desaturase gene in the sorghum genome is Sobic.002g430100, which has 75% identity with Msd3 in amino acid sequence. In many plants, Fad7 has been showed to be the major chloroplast-targeted linoleic acid desaturase while Fad8 is a cold-inducible and may compensate the function of Fad7 under certain environmental conditions (Berberich et al., supra; Li et al., supra; Roman et al., supra; Tovuu et al., supra).

From the panicle phenotype of the msd3 mutant, Msd3 could be Fad7, instead of Fad8 as annotated on Gramene. To determine whether Msd3 is Fad7 or Fad8, we constructed a matrix to identify between the two sorghum genes with Fad7 and Fad8 annotated from rice and maize (Table 2). Msd3 shows 84.8% identity with rice Fad7 (OsFAD7_LOC_Os03g18070.1) and 74.6% identity with rice Fad8 (OsFAD8_LOC_Os07g49310.1). The other linoleic acid desaturase (Sobic.002g430100) shows 76.2% identity with rice Fad7 but 81.5% with rice Fad8. Thus, Msd3 is most likely to be Fad7. Maize has three chloroplast-targeted linoleic desaturases. Based on similarity to rice genes, the gene ZmFAD7_Zm00001d047743_P001 has 84% identity with rice Fad7 but 74% identity with rice Fad8 (Table 2). Thus, this gene appears to be maize Fad7. The gene ZmFAD8_Zm00001d028742_P001 has 84.8% identify with rice Fad7 but 76% identity with rice Fad8. Thus, this gene is more closely related to rice Fad7 than to rice Fad8. Furthermore, the gene ZmFAD8_Zm00001d028742_P001 shares 93% amino acid identity with ZmFAD7_Zm00001d047743_P001. It appears that the gene ZmFAD8_Zm00001d028742_P001 is more likely another copy of Fad7, instead of Fad8. Based on this sequencing analysis, the Zm00001d007228_T001 is most likely Fad8 because it has 79% identity with rice Fad8 but 74.8% identity with rice Fad7. Furthermore, it has about 75% identity with both ZmFAD7_Zm00001d047743_P001 and ZmFAD8_Zm00001d028742_P001. Thus, we concluded that Msd3 gene is Fad7, instead of Fad8 as annotated on Gramene.

TABLE 2
Fad7/Fad8 Amino Acid Identity Matrix.

| | OsFAD7_LOC_Os03g18070.1 | ZmFAD7_Zm00001d047743_P001 | MSD3 |
|---|---|---|---|
| OsFAD7_LOC_Os03g18070.1 | 100 | 84.09 | 84.82 |
| ZmFAD7_Zm00001d047743_P001 | 84.09 | 100 | 92.74 |
| MSD3 | 84.82 | 92.74 | 100 |
| ZmFad8_Zm00001d028742_P001 | 84.75 | 92.95 | 96.18 |
| OsFAD8_LOC_Os07g49310.1 | 74.76 | 74.94 | 74.63 |
| Zm00001d007228_T001 | 74.14 | 74.2 | 74.88 |
| Sobic.002G430100.1 | 76.17 | 75.98 | 76.66 |

| | ZmFAD8_Zm00001d028742_P001 | OsFAD8_LOC_Os07g49310.1 |
|---|---|---|
| OsFAD7_LOC_Os03g18070.1 | 84.75 | 74.76 |
| ZmFAD7_Zm00001d047743_P001 | 92.95 | 74.94 |
| MSD3 | 96.18 | 74.63 |
| ZmFad8_Zm00001d028742_P001 | 100 | 76.04 |
| OsFAD8_LOC_Os07g49310.1 | 76.04 | 100 |
| Zm00001d007228_T001 | 75.12 | 79 |
| Sobic.002G430100.1 | 76.85 | 81.55 |

| | ZmFAD7_Zm00001d047743_P001 | Sobic.002G430100.1 |
|---|---|---|
| OsFAD7_LOC_Os03g18070.1 | 74.14 | 76.17 |
| ZmFAD7_Zm00001d047743_P001 | 74.2 | 75.98 |
| MSD3 | 74.88 | 76.66 |
| ZmFad8_Zm00001d028742_P001 | 75.12 | 76.85 |
| OsFAD8_LOC_Os07g49310.1 | 79 | 81.55 |
| Zm00001d007228_T001 | 100 | 90.45 |
| Sobic.002G430100.1 | 90.45 | 100 |

### Example 2

Analysis of Lipid and Jasmonic Acid Profiles of Msd3 Mutants

To analyze the effects of the various msd3 mutations on the lipid profiles, samples were treated as follows. The first matured leaf and panicles at about 3 cm long were quickly immersed in 3.0 ml 75° C. isopropanol with 0.01% BHT for 15 min and stored at −80° C. until use. Lipids were extracted and analyzed following the procedure established in the Lipidomic Center of Kansas State University (www.k-state.edu/lipid/).

To analyze the effects of the various msd3 mutations on jasmonic acid (JA) production samples were treated as follows. The first matured leaf and panicles at about 3 cm long were quickly immersed in liquid nitrogen. JAs were extracted and determined utilizing standard protocols. Briefly, for JA quantification, samples were solvent extracted, methylated, collected on a polymeric adsorbent using vapor phase extraction (VPE), and analyzed using GC/isobutene chemical ion mass spectrometry (CI-MS) as previously described (Schmelz et al., Plant J., (2004) 39:790-808). Metabolite quantification was based on d₅-JA (Sigma-Aldrich, St. Louis, Mo., USA) as an internal standard.

Mutation in Msd3 Gene Dramatically Reduced Levels of Linolenic Acid

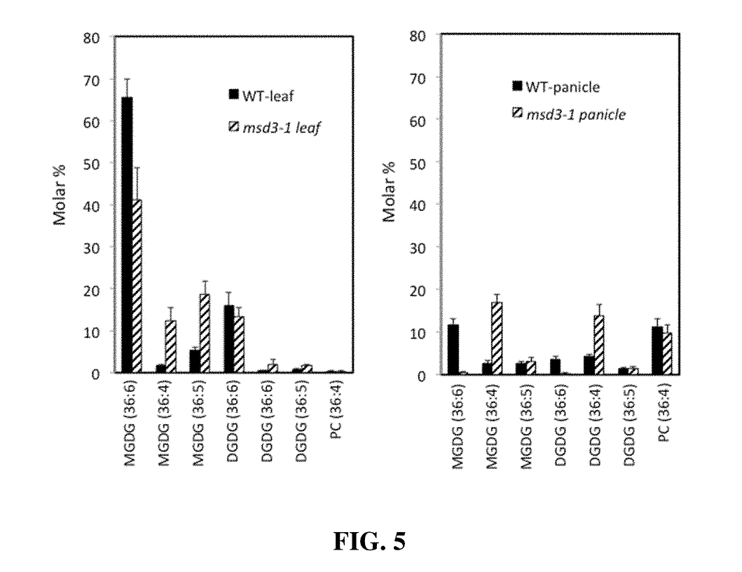

To determine the effect the msd3 on lipid composition of leaves and panicles, we determined the lipid profiles of msd3 in comparison with the WT. In WT BTx623 leaves, 18 carbon fatty acids account for over 92% of the total leaf lipids (Table 3). The 7 abundant lipid species with >1% of molar percentage were plotted in FIG. 5. Galactolipids, including MGDG and DGDG, are major lipids in leaves, accounting over 90% of the total lipids. The lipid species with 36 carbon and six double bonds (36:6) consist of two linolenic acid molecules. The lipid species with four double bonds (36:4) consist of two linoleic acid molecules. While the species with five double bonds (36:5) consist of one linoleic acid and one linolenic acid. As can be seen from FIG. 5, the mutation in Msd3 gene reduced the levels of lipid species of 36:6 and with concomitant increase in lipid species of 36:4. The ratio of linolenic acid to linoleic acid in the WT BTx623 leaves was 13.43 to 1, which was reduced to 2.48 in msd3-3 leaves. In general, panicles have much less linolenic acid. In WT BTx623 panicles, the ratio of linolenic acid to linoleic acid was 0.62, which was reduced to 0.08 in the msd3-3 mutant panicles. As a result, the msd3-3 panicle had very little linolenic acid (7% vs 38% in WT panicles). This result indicates that Msd3 is, indeed, an omega-3 fatty acid desaturase that catalyzes the desaturation of linoleic acid to linolenic acid in both leaves and panicles. The dramatic effect of msd3 mutation on the reduction of linoleic acid desaturation was consistent with Msd3 as Fad7, because Fad7 has been shown to have major effect on linoleic acid desaturation in other plant species (Li et al., 2003, supra; Roman et al., supra; Tovuu et al., supra).

TABLE 3
Lipid species in the leaves and panicles of BTx623 and msd3-3 (SBp6).

| | WT | | WT | | P6 | | P6 | |
|---|---|---|---|---|---|---|---|---|
| | leaves | | panicles | | leaves | | panicles | |
| | (% total | | (% total | | (% total | | (% total | |
| | lipid) | | lipid) | | lipid) | | lipid) | |
| Lipid species | avg | stdev | avg | stdev | avg | stdev | avg | stdev |
| PC (36:4) | 0.38 | 0.13 | 11.34 | 1.76 | 0.38 | 0.11 | 9.81 | 1.95 |
| PC (36:3) | 0.10 | 0.06 | 5.22 | 0.24 | 0.02 | 0.05 | 3.94 | 1.01 |
| DGDG (36:4) | 0.48 | 0.12 | 4.35 | 0.43 | 2.02 | 1.34 | 13.92 | 2.68 |
| PE (36:4) | 0.12 | 0.04 | 2.87 | 0.22 | 0.14 | 0.05 | 2.92 | 0.65 |
| DGDG (36:3) | 0.27 | 0.13 | 2.80 | 0.31 | 0.32 | 0.12 | 3.42 | 0.77 |
| MGDG (36:4) | 1.81 | 0.21 | 2.60 | 0.70 | 12.43 | 3.17 | 16.90 | 1.94 |
| MGDG (36:3) | 0.12 | 0.04 | 0.85 | 0.48 | 0.23 | 0.15 | 1.75 | 0.56 |
| PE (36:3) | 0.01 | 0.01 | 0.59 | 0.19 | 0.01 | 0.01 | 0.36 | 0.07 |
| PI (36:3) | 0.01 | 0.01 | 0.58 | 0.07 | 0.01 | 0.01 | 0.48 | 0.08 |
| PG (36:4) | 0.01 | 0.01 | 0.15 | 0.03 | 0.01 | 0.00 | 0.14 | 0.03 |
| PG (36:3) | 0.00 | 0.00 | 0.13 | 0.09 | 0.00 | 0.00 | 0.07 | 0.02 |
| PA (36:3) | 0.00 | 0.00 | 0.08 | 0.03 | 0.00 | 0.00 | 0.09 | 0.03 |
| PS (36:3) | 0.00 | 0.00 | 0.02 | 0.00 | 0.00 | 0.00 | 0.00 | 0.00 |
| PS (36:4) | 0.00 | 0.00 | 0.01 | 0.00 | 0.00 | 0.00 | 0.02 | 0.01 |
| PI (36:4) | 0.03 | 0.01 | 0.86 | 0.12 | 0.05 | 0.03 | 0.85 | 0.16 |
| MGDG (36:5) | 5.28 | 0.81 | 2.66 | 0.46 | 18.72 | 3.21 | 3.03 | 1.10 |
| PC (36:5) | 0.34 | 0.09 | 2.42 | 1.76 | 0.18 | 0.17 | 1.24 | 1.25 |
| DGDG (36:5) | 0.73 | 0.19 | 1.50 | 0.27 | 3.24 | 1.44 | 1.51 | 0.46 |
| PE (36:5) | 0.10 | 0.05 | 0.52 | 0.11 | 0.05 | 0.01 | 0.27 | 0.03 |
| PI (36:5) | 0.03 | 0.01 | 0.12 | 0.03 | 0.03 | 0.01 | 0.06 | 0.03 |
| PG (36:5) | 0.00 | 0.00 | 0.02 | 0.02 | 0.00 | 0.00 | 0.04 | 0.02 |
| LPE (18:3) | 0.00 | 0.00 | 0.01 | 0.02 | 0.00 | 0.00 | 0.01 | 0.01 |
| MGDG (36:6) | 65.51 | 4.48 | 11.76 | 1.31 | 41.20 | 7.72 | 0.47 | 0.31 |
| DGDG (36:6) | 15.93 | 3.32 | 3.74 | 0.54 | 13.14 | 2.03 | 0.26 | 0.17 |
| PC (36:6) | 0.09 | 0.06 | 0.15 | 0.29 | 0.00 | 0.00 | 0.31 | 0.43 |
| PE (36:6) | 0.02 | 0.02 | 0.01 | 0.01 | 0.01 | 0.01 | 0.01 | 0.02 |
| PG (36:6) | 0.00 | 0.01 | 0.00 | 0.01 | 0.00 | 0.00 | 0.00 | 0.00 |
| PI (36:6) | 0.14 | 0.07 | 0.00 | 0.01 | 0.12 | 0.04 | 0.00 | 0.00 |
| PS (36:6) | 0.00 | 0.00 | 0.00 | 0.00 | 0.00 | 0.00 | 0.00 | 0.00 |
| PA (36:6) | 0.00 | 0.00 | 0.00 | 0.00 | 0.00 | 0.00 | 0.00 | 0.00 |
| PC (34:2) | 0.60 | 0.17 | 11.23 | 1.82 | 0.44 | 0.27 | 10.02 | 2.01 |
| PE (34:2) | 0.29 | 0.06 | 4.43 | 0.65 | 0.23 | 0.04 | 4.04 | 0.65 |
| PI (34:2) | 0.37 | 0.07 | 4.28 | 0.95 | 0.39 | 0.06 | 4.26 | 0.42 |
| 18:3/18:2 Ratio | 13.43 | | 0.62 | | 2.48 | | 0.08 | |

Mutations in Msd3 May Lead to Msd Phenotype Through Reduction of JA

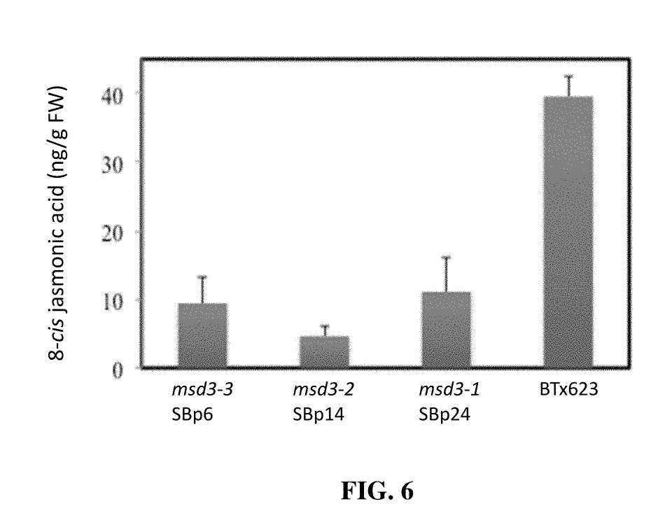

We propose that the panicle phenotype of msd3 mutants may be due to the reduction of linolenic acid (18:3), which is the substrate for JA biosynthesis (Lyons et al., supra). Thus, we determined JA levels in the leaves and panicles of BTx623 and three msd3 mutant alleles. As shown in FIG. 6, cis-JA, the biologically active JA, was reduced in all three msd3 lines compared with BTx623. The JA levels in panicles were below detectable limit in both BTx623 and msd3 mutants. In general, sorghum tissues have low JA comparing with other plants (Creelman & Mullet, Proc. Nat'l. Acad. Sci. USA, (1995) 92:4114-9). Considering the low linolenic acid level in WT panicles, about 38% comparing with over 90% in leaves, it is not unremarkable that the JA level was below detectable levels in the tested panicles. Comparing with the leaves of BTx623 WT, the leaves of msd3-1 contained only 7% linolenic acid. Based on the reduction of JA in the leaves of msd3 mutants, it could be inferred that the JA level in msd3 panicles was also reduced. Thus, we concluded that reduction in JA due to the decreased level linolenic acid in msd3 panicles led the msd3 panicle phenotype.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A genetically altered plant or parts thereof, wherein said plant comprises a mutation in a gene encoding a plastid-targeted ω-3 fatty acid desaturase having a nucleic acid sequence at least 85% identical to SEQ ID NO: 4, SEQ ID NO: 6, SEQ ID NO: 8, SEQ ID NO: 10, SEQ ID NO: 12, SEQ ID NO: 13, SEQ ID NO: 14, or SEQ ID NO: 15, wherein said mutation causes a reduction in the enzymatic activity of said plastid-targeted ω-3 fatty acid desaturase compared to the enzymatic activity of a wild-type plant.

2. The genetically altered plant of claim 1, wherein said genetically altered plant has reduced amount of 8-cis-jasmonic acid in its leaf tissue compared to the amount of 8-cis-jasmonic acid in leaf tissue of a non-genetically altered plant.

3. The genetically altered plant of claim 1, wherein the plant is a cereal crop plant selected from the group consisting of sorghum, maize, rice, barley, oats, wheat, rye, millet, and triticale.

4. The genetically altered plant of claim 1, wherein said mutation is present in two copies in the genetically altered plant.

5. The genetically altered plant of claim 1, wherein the plant part is a plant cell.

6. The genetically altered plant of claim 1, wherein the plant part is a seed.

7. The genetically altered plant of claim 1, wherein the plant part is pollen.

8. A genetically altered plant or parts thereof, wherein said plant comprises a null mutation in at least two copies of a gene encoding a plastid-targeted ω-3 fatty acid desaturase having a nucleic acid sequence that encodes a protein at least 80% identical to SEQ ID NO: 2.

9. The genetically altered plant of claim 8, wherein said genetically altered plant has reduced amount of 8-cis-jasmonic acid in its leaf tissue compared to the amount of 8-cis-jasmonic acid in leaf tissue of a non-genetically altered plant.

10. The genetically altered plant of claim 8, wherein the plant is a cereal crop plant selected from the group consisting of sorghum, maize, rice, barley, oats, wheat, rye, millet, and triticale.

11. The genetically altered plant of claim 8, wherein the plant part is a plant cell.

12. The genetically altered plant of claim 8, wherein the plant part is a seed.

13. The genetically altered plant of claim 8, wherein the plant part is pollen.

14. A method for constructing a genetically altered cereal crop that has the MSD3 phenotype, the method comprising:
(i) introducing an altered msd3 nucleic acid into a cereal crop or grass plant to provide a genetically altered cereal crop or grass plant;
(ii) selecting the genetically altered cereal crop that is homozygous for the altered msd3 nucleic acid, thereby constructing the genetically altered cereal crop or grass plant, wherein the genetically altered cereal crop or grass plant has the MSD3 phenotype.

15. The method of claim 14, wherein the cereal crop is selected from the group consisting of sorghum, maize, rice, barley, oats, wheat, rye, millet, and triticale.

16. The method of claim 14, wherein the cereal crop is sorghum and the altered msd3 nucleic acid is selected from the group consisting of SEQ ID NO: 4, SEQ ID NO: 6, SEQ ID NO: 8, SEQ ID NO: 10, SEQ ID NO: 12, SEQ ID NO: 13, SEQ ID NO: 14, or SEQ ID NO: 15.

17. The method of claim 14, wherein said introducing said altered msd2 nucleic acid occurs via introgression, genomic editing, or exposing said cereal crop or grass plant to a mutagen, and said selecting said genetically altered cereal crop or grass plant occurs via marker assisted selection.

18. The method of claim 14, wherein the MSD3 phenotype arises from a mutation in a plastid-targeted co-3 fatty acid desaturase and wherein the genetically altered cereal crop has a reduced amount of 8-cis-jasmonic acid in its leaf tissue compared to the amount of 8-cis-jasmonic acid in leaf tissue of a non-genetically altered plant.
